(19) United States
(12) Patent Application Publication
(10) Pub. No.: US 2013/0245655 A1
(43) Pub. Date: Sep. 19, 2013

(54) CONCEALED BLADE SCALPEL

(71) Applicant: Sudha S. Mahurkar, Aurora, IL (US)

(72) Inventor: Sakharam D. Mahurkar, Aurora, IL (US)

(21) Appl. No.: 13/758,541

(22) Filed: Feb. 4, 2013

Related U.S. Application Data
(60) Provisional application No. 61/608,725, filed on Mar. 9, 2012.

(51) Int. Cl. A61B 17/3211

(52) U.S. Cl. CPC A61B 17/3211
USPC 606/167

(57) ABSTRACT

A concealed-blade scalpel includes an elongated housing forming an interior cavity and an opening at one end of the housing to provide access to the interior cavity. A surgical cutting blade is mounted within the cavity for sliding movement relative to the housing so that the blade can be moved between (1) a retracted position in which the cutting edge of the blade is located entirely within the cavity, and (2) an advanced position in which the cutting edge of the blade is positioned outside the housing to permit the blade to be used for cutting. An actuator is coupled to the blade for manually moving the blade to the advanced position in response to manual pressure applied to the actuator, and a biasing element continuously urges the blade toward the retracted position for automatically retracting the blade.

### CROSS-REFERENCE TO RELATED APPLICATIONS

This application claims priority to U.S. Provisional Application Ser. No. 61/608,725 filed Mar. 9, 2012, which is hereby incorporated by reference herein in its entirety

### FIELD OF THE INVENTION

This invention generally relates to scalpels and lancets, which are small and extremely sharp-bladed instruments used in the medical field for performing surgical procedures on patients and for anatomical dissection. These instruments are extensively used in procedures performed in operating rooms as well as in clinical settings. They are also used in arts, crafts, box cutting and various everyday applications. They are intended for cutting, incising, stabbing or enucleating body tissues, depending on the shape of the sharp edge of the blade. Scalpels are typically single-piece structures consisting of a sharp blade, usually made of hardened and tempered stainless steel or high carbon steel, reversibly or permanently attached to a rigid elongated handle that permits manipulation by the user to perform the desired procedures. Blades with permanently attached handles are usually disposable, while handles with removably attached blades are typically re-usable with fresh blades. Disposable scalpels usually have a plastic handle with a blade, and the entire instrument is discarded after a single use. Re-usable scalpels can have attached re-sharpenable blades or, more commonly, removable and replaceable blades. Double-edged scalpels are referred to as “lancets.”

A disposable scalpel having a retractable blade is disclosed in Haining U.S. Pat. No. 5,330,493. That scalpel has a blade with a bifurcated body installed inside the handle; the blade is detachable and replaceable and held in position by diverging legs. The blade can be positioned in exposed, intermediate, retracted and locked position.

Another scalpel with a spring-loaded, automatically retracted blade is disclosed in Platts U.S. Pat. No. 5,403,337. The Platts scalpel has a detachable and retractable blade. The scalpel has two channels, one for a spring that runs along the entire length and another for the blade. Both the spring and the blade are detachable and removable. The blade is accessible through side windows, and there are tabs that permit the localization of the blade wherever it is necessary. It has two tabs 20 near the front end and another tab 22 near the rear end, both on the same surface. A spring 26 extends from the front end and pushes the blade backwards by engaging a step on the blade. The spring, which constantly biases the blade inside the handle, is a compression spring that must be further compressed or shortened to advance the blade. Latches maintain the spring at different locations.

Another retractable knife is disclosed in Knoop U.S. Pat. No. 4,805,304 which shows an oval knife split in the middle into two halves. Each half has a central semi-circular channel carved out to form space to lodge the blade; the channel is open at the front end, for exposing the blade for cutting, while the other end of the channel is blind. Near the front end, the sides of the knife body have two oval windows on each side to expose both sides of the blade, and two concave switches are fastened to the sides of the blade. When advanced, the two concave switches are expected to advance the sharp end of the blade through the front end of the knife. The Knoop cites Anderson U.S. Pat. No. 2,862,296 as teaching a similar enclosure of a blade in the center of two halves of a knife. The blade is expected to be advanced through a window on the side for accessing a switch attached to the blade and another window on the back side. Knoop teaches that windows to advance the blade and to access the switch must be present on both sides and both halves of the blade. When an extension spring is used and relied on to advance a blade by stretching, its anchored end and anchor must be established; otherwise an effort to advance the blade can cause the entire knife to slip out and fall off the handle. Special features to move the knife backwards, while the blade is advanced forward, must be provided. It is also essential that these two counter-acting forces be applied at the same location.

Other examples of safety scalpels are disclosed in U.S. Pat. Nos. 7,900,362, 7,857,824, 7,669,337, 7,153,317, 6,979,340, 6,757,977, 6,629,985, 6,589,258, 6,022,364, 5,868,771, 5,779,724, 5,571,127, 5,569,282, 5,423,843, 5,417,704, 5,342,379, 5,330,492 and 5,292,329.

### SUMMARY

According to one embodiment, a concealed-blade scalpel includes an elongated housing forming an interior cavity and an opening at one end of the housing to provide access to the interior cavity. A surgical cutting blade is mounted within the cavity for sliding movement relative to the housing so that the blade can be moved between (1) a retracted position in which the cutting edge of the blade is located entirely within the cavity, and (2) an advanced position in which the cutting edge of the blade is positioned outside the housing to permit the blade to be used for cutting. A biasing element is mounted within the cavity and coupled to the blade and the housing for urging the blade toward the retracted position. An actuator is coupled to the blade for manually moving the blade to the advanced position in response to manual pressure applied to the actuator, and the biasing element continuously urges the blade toward the retracted position so that the blade is automatically moved to the retracted position when the manual pressure applied to the actuator is removed or reduced.

### BRIEF DESCRIPTION OF THE DRAWINGS

The invention will be better understood from the following description of preferred embodiments together with reference to the accompanying drawings, in which:

FIG. 1 is a front perspective view of a surgical scalpel with its blade in a fully advanced position.

FIG. 2 is an exploded front perspective view of the scalpel shown in FIG. 1, with the hinged portion of its housing open.

FIG. 3 is an exploded front perspective of the scalpel shown in FIG. 1, from the opposite side of the scalpel, with the hinged portion of its housing open.

FIG. 4A is an enlarged side elevation of the distal portion of the housing of the scalpel of FIG. 1, with the hinged portion of the housing open.

FIG. 4B is a top plan view of the scalpel of FIG. 1.

FIG. 5 is an enlarged section taken along line 5-5 in FIG.4B.

FIG. 6 is an enlarged section taken along line 6-6 in FIG. 4B.

FIG. 7 is a further enlarged section taken along line 7-7 in FIG. 4B.

FIG. 8 is an enlarged section taken along line 8-8 in FIG. 4B.

FIG. 9 is an enlarged section taken along line 9-9 in FIG. 4B.

FIG. 10 is an enlarged section taken along line 10-10 in FIG. 4B.

FIG. 11 is a front perspective view of a modified surgical scalpel with its blade fully retracted.

FIG. 12 is an exploded front perspective view of the scalpel shown in FIG. 11, with the hinged portion of its housing open.

FIG. 13 is an exploded front perspective of the scalpel shown in FIG. 11, from the opposite side of the scalpel, with the hinged portion of its housing open.

FIG. 14 is a top plan view of a left-hand end portion the scalpel of FIG. 11.

FIG. 15 is an end elevation of the scalpel of FIG. 11, taken from the left-hand end as viewed in FIG. 11.

FIG. 16 is a side elevation of the portion of the scalpel shown in FIG. 14.

FIG. 17 is an enlarged section taken along the line 17-17 in FIG. 15, with the actuator in its retracted position.

FIG. 18 is the same sectional view shown in FIG. 17, with the actuator moved to a partially advanced position.

FIG. 19 is the same sectional view shown in FIG. 17, with the actuator moved to its fully advanced position.

FIG. 20 is an exploded front perspective from one side of another modified surgical scalpel.

FIG. 21 is an exploded perspective from the opposite side of the scalpel shown in FIG. 20.

FIG. 22 is an enlarged longitudinal section taken through the middle of a portion of the scalpel of FIGS. 20 and 21.

FIG. 23 is a longitudinal section taken through the same portion of the scalpel shown in FIG. 22 but laterally offset from the section shown in FIG. 22.

FIG. 24 is the same exploded perspective shown in FIG. 21 but with a different serpentine element.

FIG. 25 is an enlarged longitudinal section taken along one side of the serpentine element in the scalpel shown in FIG. 24.

FIG. 26 is a further enlarged longitudinal section, orthogonal to the section shown in FIG. 25, taken through the center of one end portion of the serpentine element in the scalpel shown in FIG. 24.

### DETAILED DESCRIPTION OF PREFERRED EMBODIMENTS

Although the invention will be described in connection with certain preferred embodiments, it will be understood that the invention is not limited to those particular embodiments. On the contrary, the invention is intended to cover all alternatives, modifications, and equivalent arrangements as may be included within the spirit and scope of the invention as defined by the appended claims.

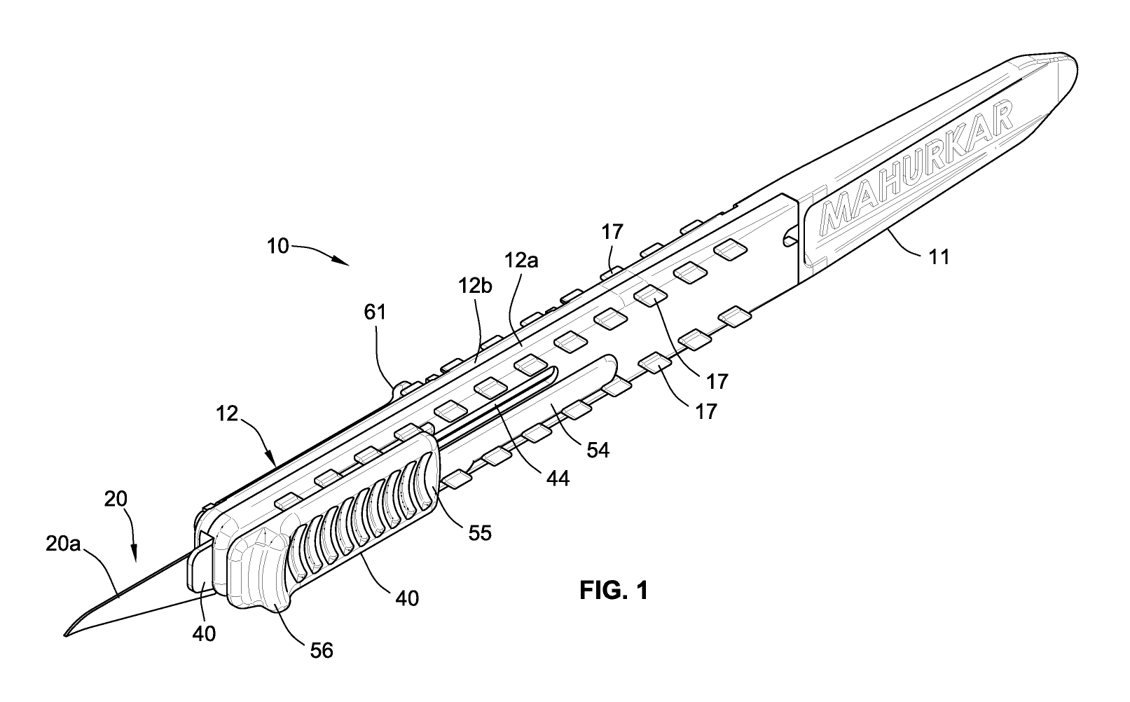

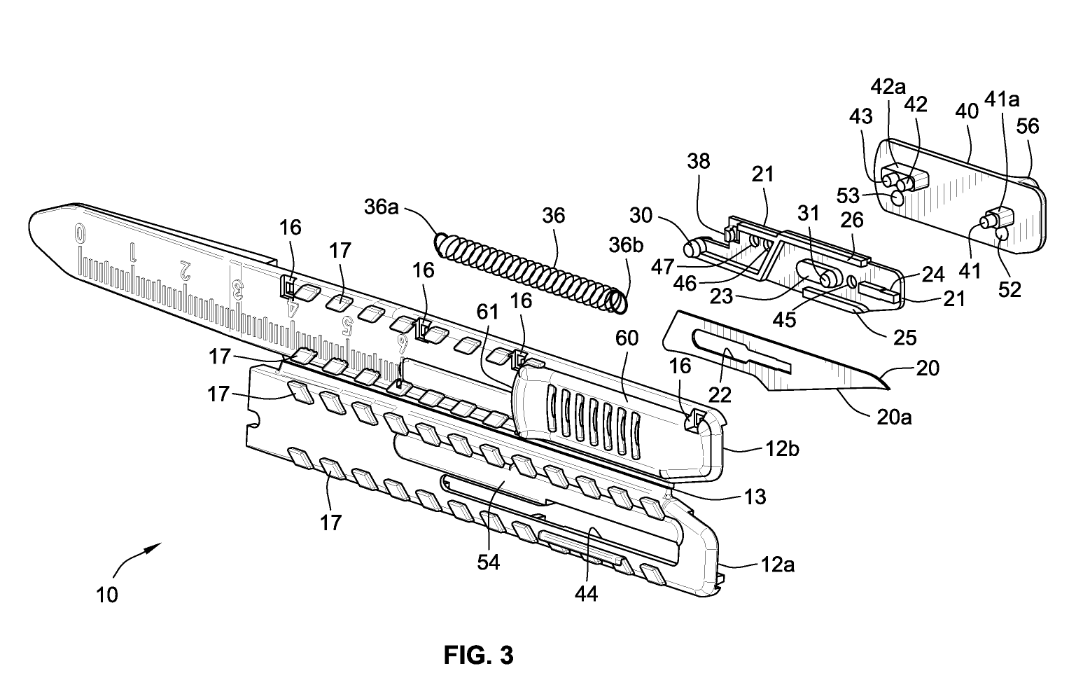

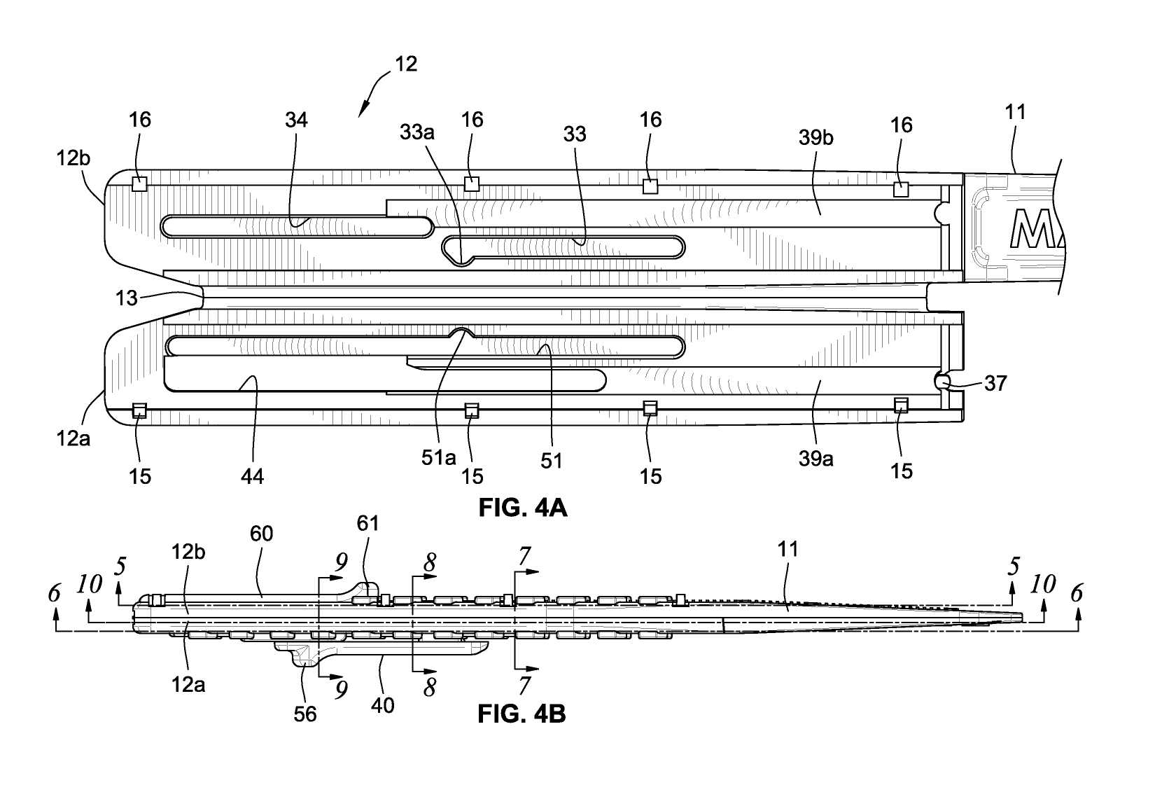

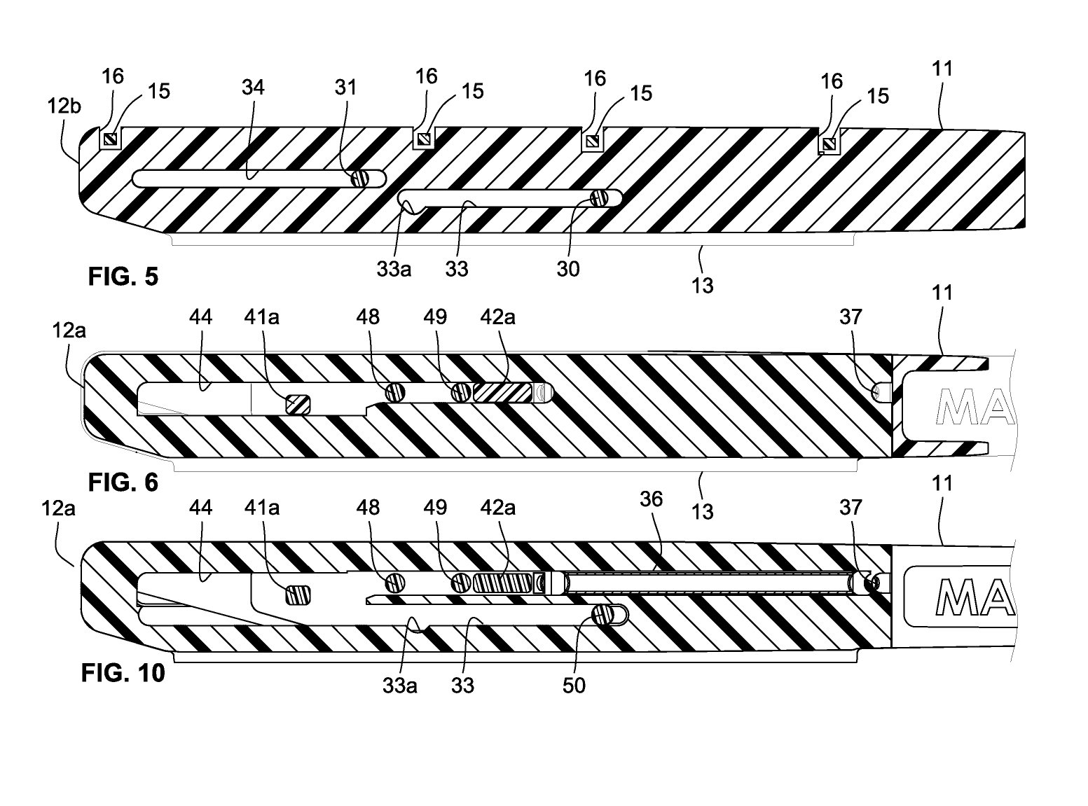

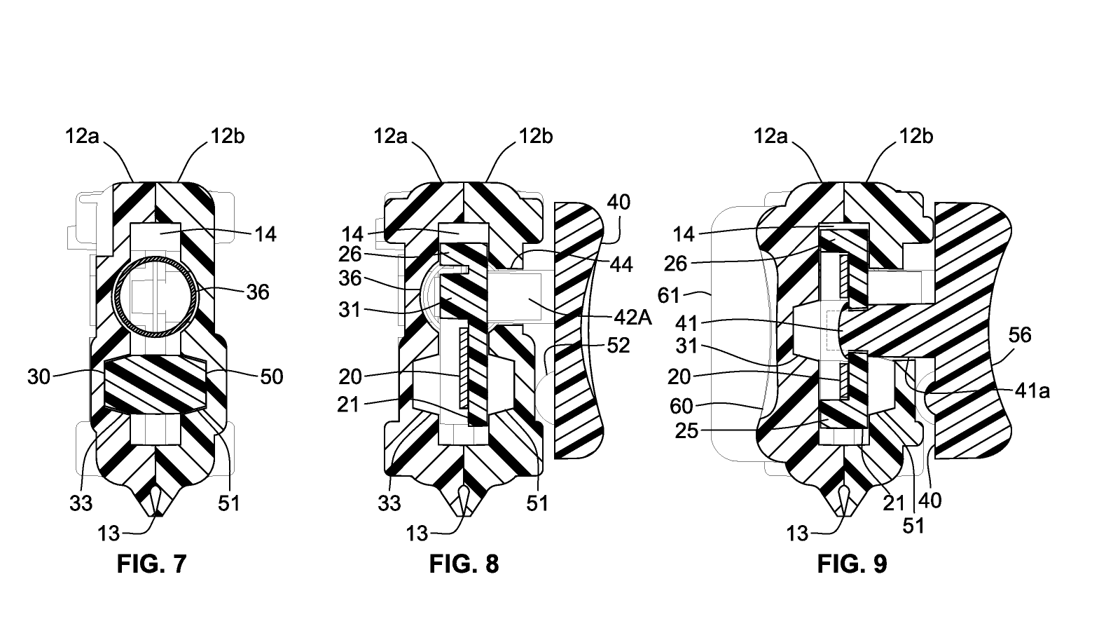

Turning now to the drawings, FIGS. 1-5 illustrate a scalpel having a molded plastic housing 10 with a rigid proximal portion 11 and a distal portion 12 that contains a surgical cutting blade 20. The distal portion 12 of the housing 10 is divided into first and second halves 12a and 12b, joined by a living hinge 13 so that the first half 12a can be pivoted relative to the second half 12b for opening and closing an internal cavity 14 (see FIGS. 7-9). The cavity 14 is formed by recesses in the opposed surfaces of the two sections 12a and 12b when the two halves 12a and 12b are in their closed positions depicted in FIGS. 1, 4B and 6-10.

When the two halves 12a and 12b are folded against each other, multiple pins 15 spaced along the free long edge of the first half 12a fit into mating holes 16 spaced along the free longitudinal edge of the second half 12b. The pins 15 can be locked in the holes 16 by a variety of different techniques, such as mechanical locking via detents formed by the surfaces of the pins and the side walls of the holes, heat staking the pins 15 to the second half 12a of the housing, adhesive bonding, etc. It is preferable that the locking of the two halves of the housing be irreversible, to prevent access to the sharp blade 20 when it is retracted inside the housing 10, as described in detail below.

To facilitate gripping the scalpel anywhere along the distal portion 12 of the housing, a series of angled projections 17 are formed along both the top and bottom edges of both sides of the distal portion 12 of the housing.

Within the cavity 14, the blade 20 is fastened to a carrier 21 configured for longitudinal sliding movement in the cavity 14. The main body of the blade 20 forms an elongated slot 22 that fits over a pair of ribs 23 and 24, and between a second pair of ribs 25 and 26, on one side of the carrier 21. The outer surfaces of the four ribs 23-26 are thermally deformed to slightly overlap the surface of the blade 20, to stake the blade to one side of the carrier 21. Thus, the blade 20 is integrally joined with the carrier 21 so that the blade 20 moves along with the carrier 21 during sliding movement of the carrier 21 within the cavity 14. The cutting edge 20a of the blade 20 projects distally beyond the distal end of the carrier 21 so that when the carrier is advanced to its most distal position, the cutting edge of the blade 20 projects beyond the distal end of the housing 10 so that the blade can be used for cutting. Conversely, when the carrier 21 is retracted to its most proximal position, the blade 20 is retracted entirely within the cavity 14 so that the blade cannot cause any accidental cuts during handling.

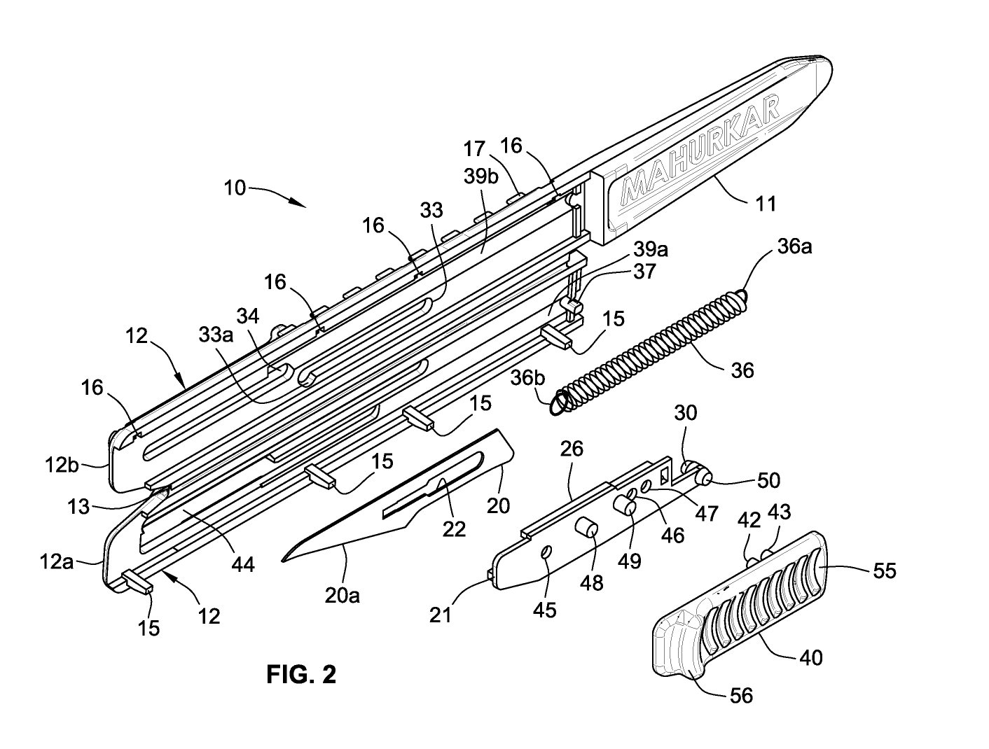

To permit guided sliding movement of the carrier 21, a pair of guide pins 30 and 31 (see FIGS. 2, 3 and 5) project laterally form one side of the carrier and ride in a corresponding pair of guide channels 33 and 34, respectively, in the cavity wall formed by the housing section 12b. The ends of the channels 33 and 34 determine the locations of the fully retracted and fully advanced positions of the carrier 21, i.e., the carrier locations where the ends of the channels 33 and 34 are engaged by the respective pins 30 and 31 of the carrier 21. The guide channel 33 has a notch 33a at the distal end of the channel so that the user feels a “click” when the carrier 21 reaches its most advanced position.

The carrier 21 is continuously urged toward its retracted position by a continuous retracting biasing force exerted on the carrier by a coil spring 36, so the carrier 21 can be advanced only by the application of an external manual force that overcomes the biasing force of the spring 36. As soon as that manual force is reduced below that of the spring 36, the carrier 21 is quickly and automatically retracted by the biasing force exerted on the carrier 21 by the spring. The spring 36 is located within the cavity 14 with the proximal end of the spring 36 attached to the proximal section 11 of the housing 10, and the distal end of the spring 36 attached to the carrier 21. Specifically, closed loops 36a and 36b on the proximal and distal ends, respectively, of the spring 36 are looped over respective hooks 37 and 38 on the housing section 12a and the carrier 21.

The spring 36 is contained in a cylindrical cavity formed by a pair of matching semi-cylindrical recesses 39a and 39b formed in the hinged housing sections 12a and 12b, respectively. The spring 36 is always partially extended when attached to the two hooks 37 and 38, so that the spring continuously biases the carrier 21 toward its retracted position, regardless of the position of the carrier along its permitted range of longitudinal movement. Even when the carrier 21 is in its fully retracted position the spring 36 biases the carrier 21 toward its retracted position, by urging the carrier pins 30 and 31 against the proximal ends of the guide slots 33 and 34.

Advancing movement of the carrier 21, toward the distal end of the housing 10, is effected by manually applying pressure to a sliding actuator 40 attached to one side of the carrier by three pins 41-43 formed by the actuator 40 and extending through a slot 44 in the housing section 12a. The three pins 41-43 fit snugly into mating holes 45-47 in the carrier 21, and can be thermally staked to the carrier. The pins 41-43 are formed on the ends of a pair of guide lugs 41a and 42a that ride within the slot 44. The proximal portion of the slot 44 is narrower than the distal portion, is only slightly wider than the width of the guide lug 42a, and extends along the full length of travel of the lug 42a. The lug 42a abuts the proximal end of the slot 44 at the same retracted limit position at which the carrier pins 30 and 31 abut the proximal ends of their respective channels 33 and 34. The guide lug 41a is spaced distally from the lug 42a and abuts the distal end of the slot 44 at the same advanced limit position at which the carrier pins 30 and 31 abut the distal ends of their respective channels 33 and 34.

A pair of guide pins 48 and 49 formed by the carrier 21 also extend into the slot 44, and a third guide pin 50 rides in a channel 51 formed in the interior surface of the housing section 12a. The pin 50 is directly opposite the pin 30 on the carrier, and the channel 51 includes a notch 51a at the same longitudinal location as the notch 33a in the channel 33. The pin 50 abuts the proximal end of the channel 51 at the same retracted limit position at which the carrier pins 30 and 31 abut the proximal ends of their respective channels 33 and 34.

The actuator 40 slides on the exterior surface of the housing section 12a, guided by the walls of the slot 44 and the channels 33-35 and 51, with the proximal ends of the channels 34, 35 and 51 defining the limit for retracting movement of the actuator 40, and the distal ends of the channels 33 and 34 defining the limit for advancing movement of the actuator 40. A pair of beads 52 and 53 formed on the bottom of the actuator 40 ride on a rail 54 formed on the exterior surface of the housing section 21a along one edge of the slot 44.

The top of the sliding actuator 40 has a serrated surface 55 to facilitate gripping the scalpel, and the distal end of the serrated surface 55 has a raised lip 56 to facilitate pushing the actuator distally toward its most advanced position, against the retracting force exerted on the carrier 21 by the biasing spring 36. A similar stationary gripping surface 60 is molded into the distal end portion of the housing section 12b, with a raised lip 61 positioned at the proximal end of the gripping surface 60 rather than the distal end. When the user slides the movable actuator 40 forwardly, e.g., by pressing his or her thumb against the lip 56, the stationary gripping surface 60 on the opposite side of the scalpel can be pressed in the opposite (retracting) direction by pressing a finger against the lip 61. This permits the user to precisely control the scalpel during a cutting operation, while maintaining the pressure required to keep the blade in its advanced position.

When the advancing pressure applied to the actuator 40 is removed or reduced below the level of the retracting force applied by the spring 30, the retracting force applied to the carrier 21 by the spring 36 pulls the carrier 21 to its retracted position and then holds the carrier in that position. Thus, if a user of the scalpel pushes the sliding actuator 40 forward to move the blade 20 to its most advanced position, for use in a cutting operation, and then releases the actuator 40 when the cutting operation is completed, the spring 36 immediately pulls the carrier 21 and thus the blade 20 to their fully retracted positions, concealing the blade 20 entirely within the housing 10. This is a safety feature that permits the blade 20 to be in its advanced position only as long as advancing pressure is applied to the actuator 40, and automatically retracting the blade as soon as that pressure is released, without any further action by the user.

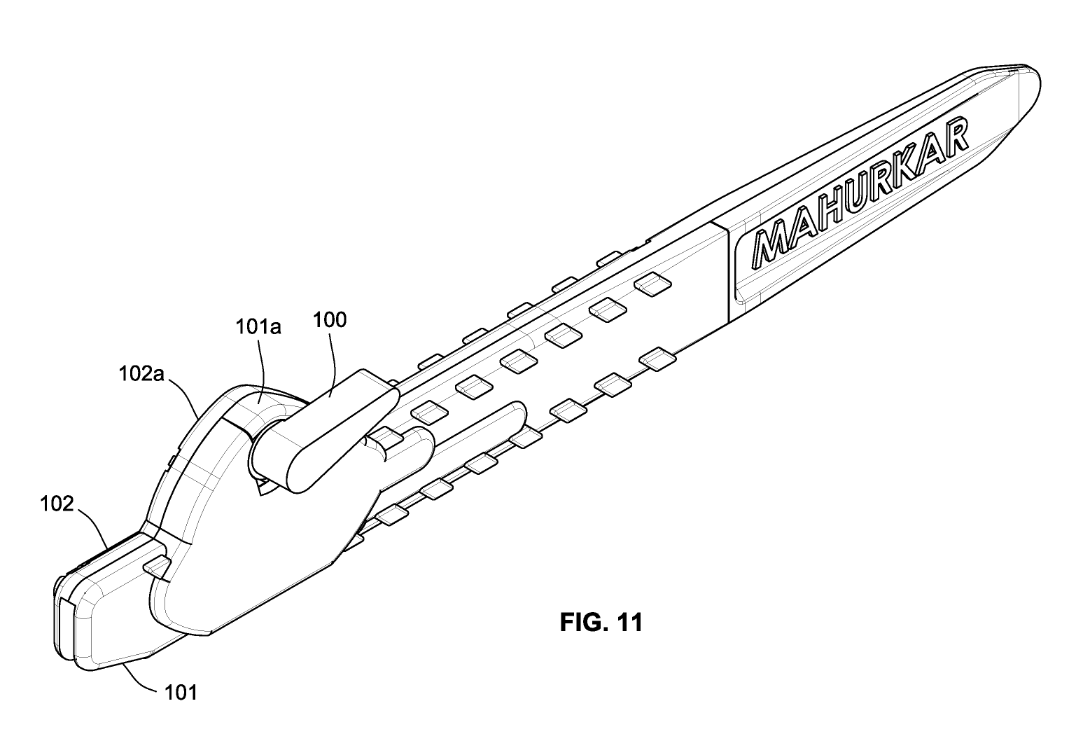

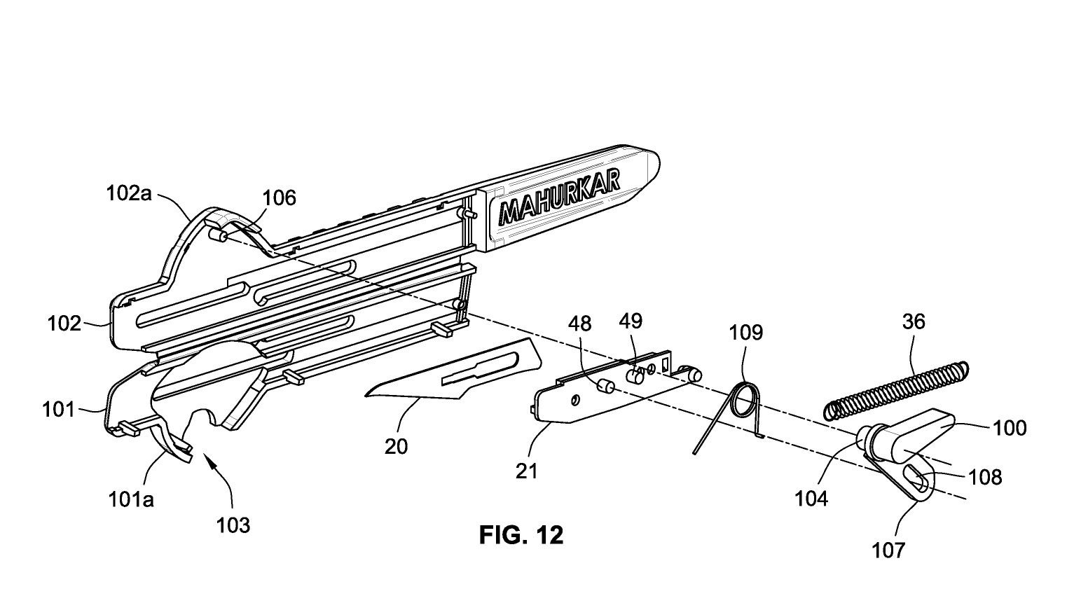

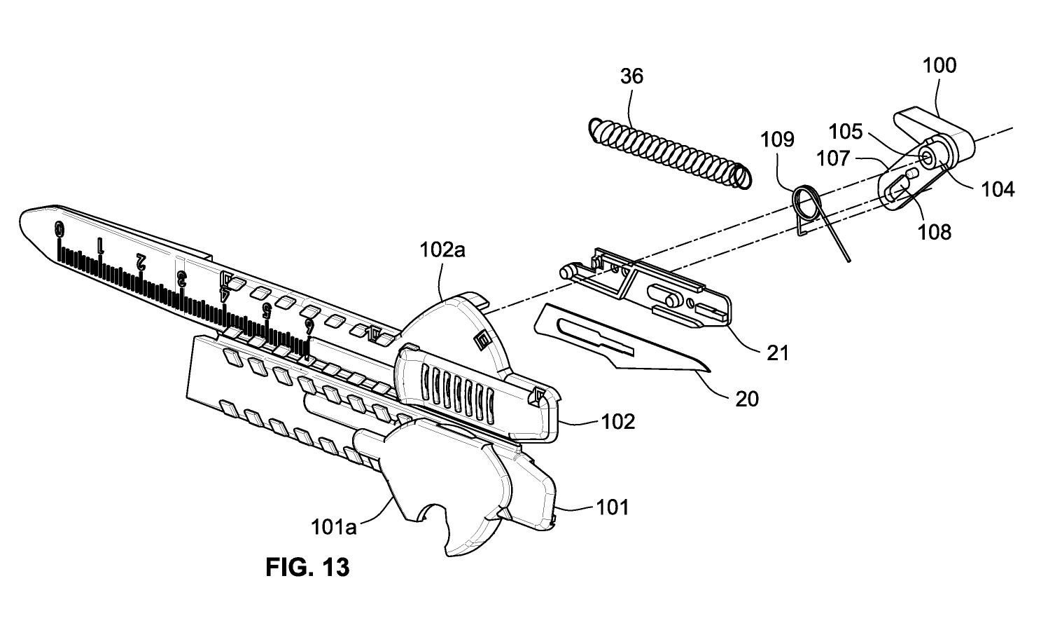

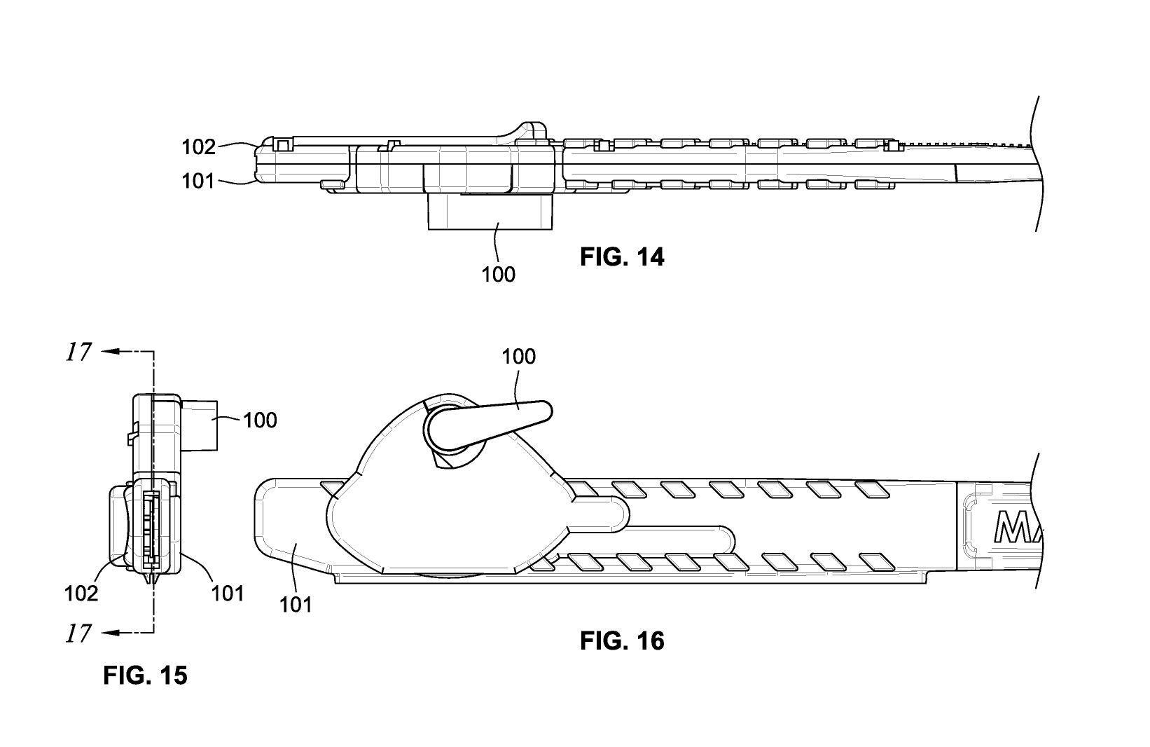

The sliding actuator 40 is a particularly appropriate actuator for use in surgical procedures in which the surgeon grips the scalpel with a “palm” grip. Other embodiments, to be described below, are more appropriate for other types of grips, such as the “fingertip” grip and the “pencil” grip. For example, the embodiment illustrated in FIGS. 11-19 utilizes a pivoting actuator 100 that can be conveniently manipulated with the surgeon's index finger when a “fingertip” grip is used. Except for the actuator and the two hinged housing sections, the structure and operation of this embodiment is the same as that described above in connection with FIGS. 1-10. The hinged housing sections 101 and 102 in the embodiment of FIGS. 11-19 include arched extensions 101a and 102a that protrude upwardly to form a cavity with an aperture 103 on one side for receiving the pivoting actuator 100. The actuator 100 includes a cylindrical portion 104 that extends through the aperture 103 and forms a recess 105 that fits over a pin 106 to permit pivoting movement of the actuator 100 about the axis of the pin 106.

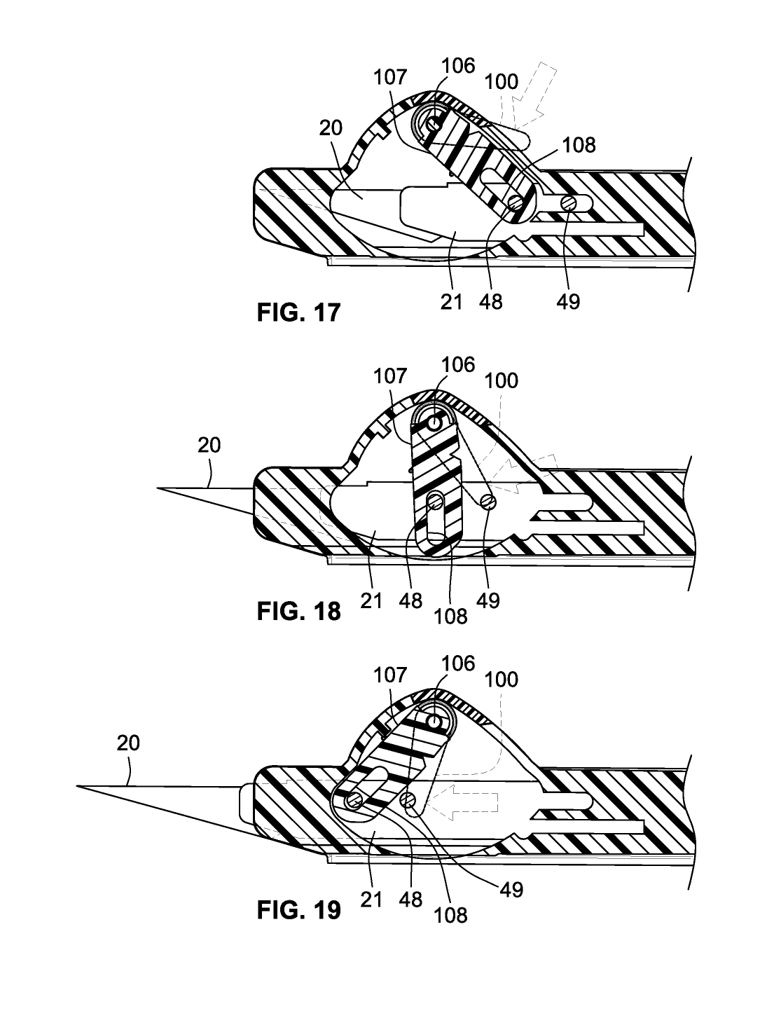

To connect the actuator 100 to the carrier 21 that carries the scalpel blade 20, a link 107 extending downwardly from the cylindrical portion 104 forms a slot 108 that fits over the pin 48 on the carrier 21. The actuator 100 is biased to its uppermost position (FIGS. 16 and 17) by a spring 109. When the actuator 100 is manually pivoted downwardly around the axis of the pin 106, in a clockwise direction as viewed in FIGS. 17-19, the walls of the slot 108 cam the carrier 21 longitudinally to advance the carrier 21 and the blade 20 to their advanced positions where the blade 20 is exposed for use in a cutting procedure. When manual pressure is removed from the actuator 100, the spring 109 returns the actuator 100 to its uppermost position, and the retracting spring 36 retracts the carrier 21, and thus the blade 20, in the same manner described above in connection with FIGS. 1-10.

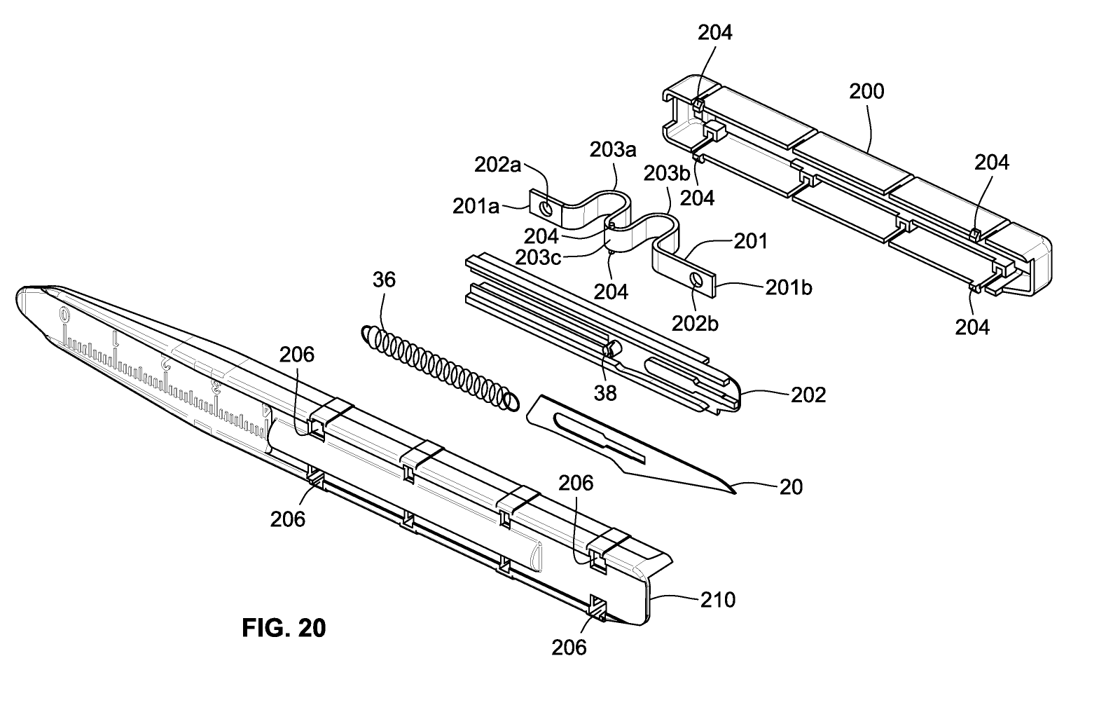

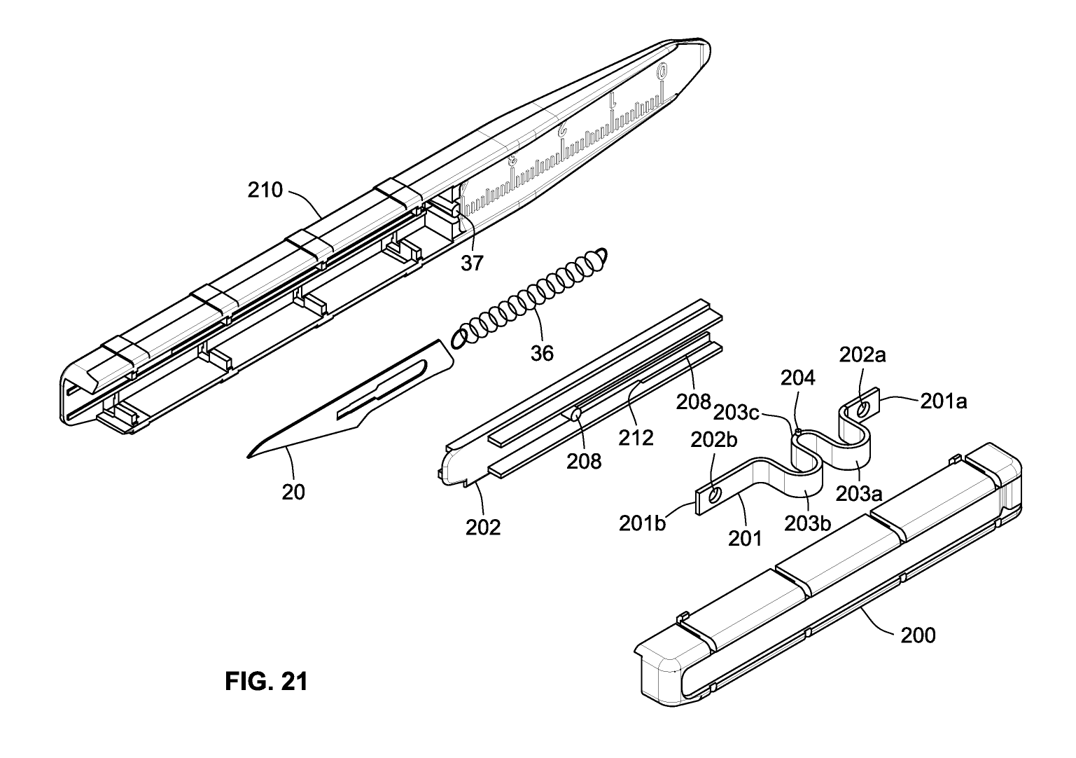

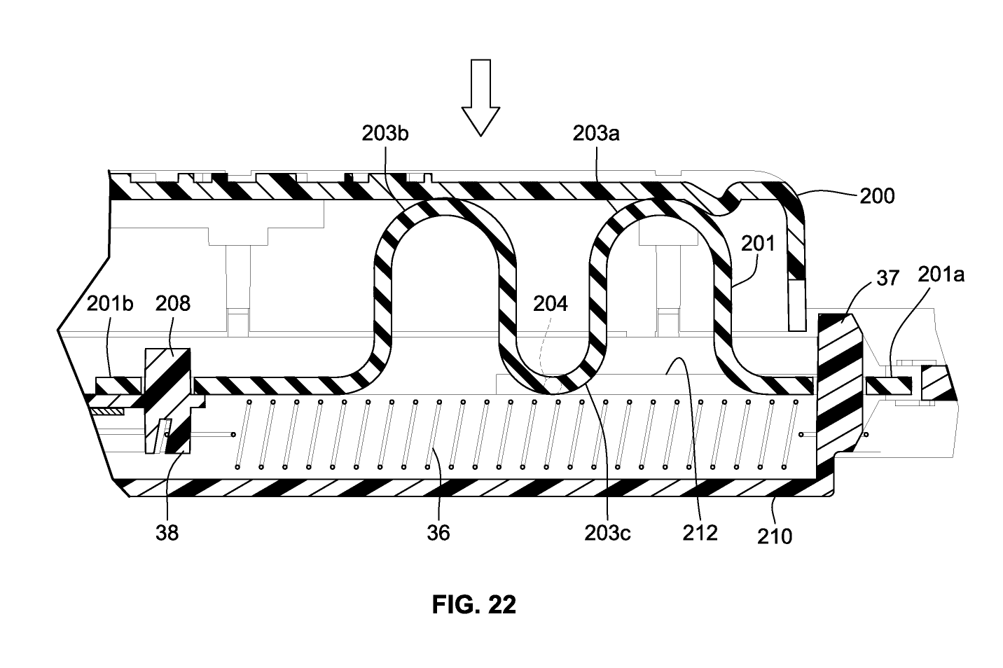

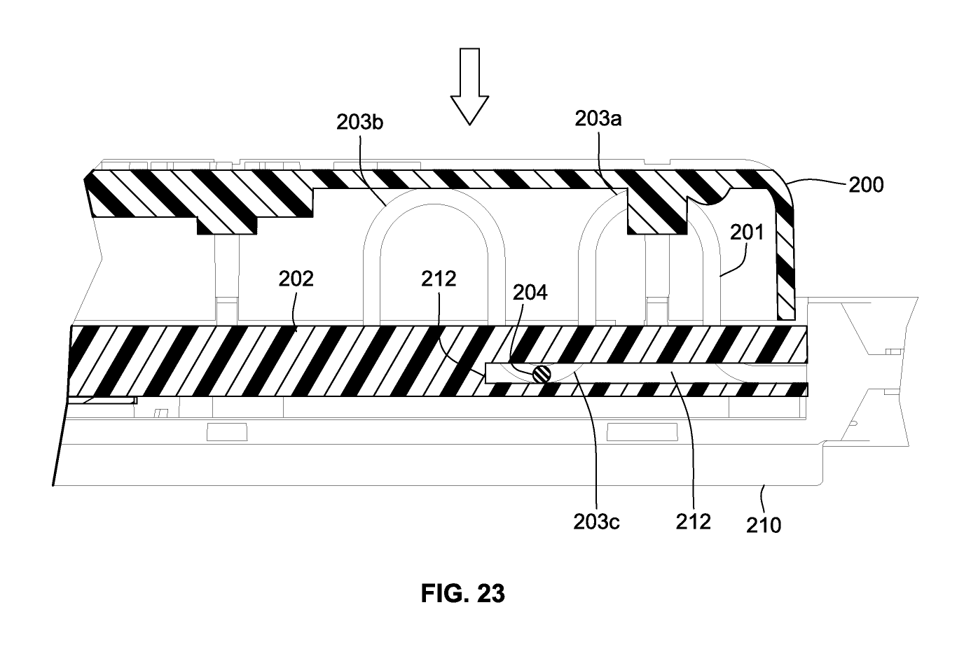

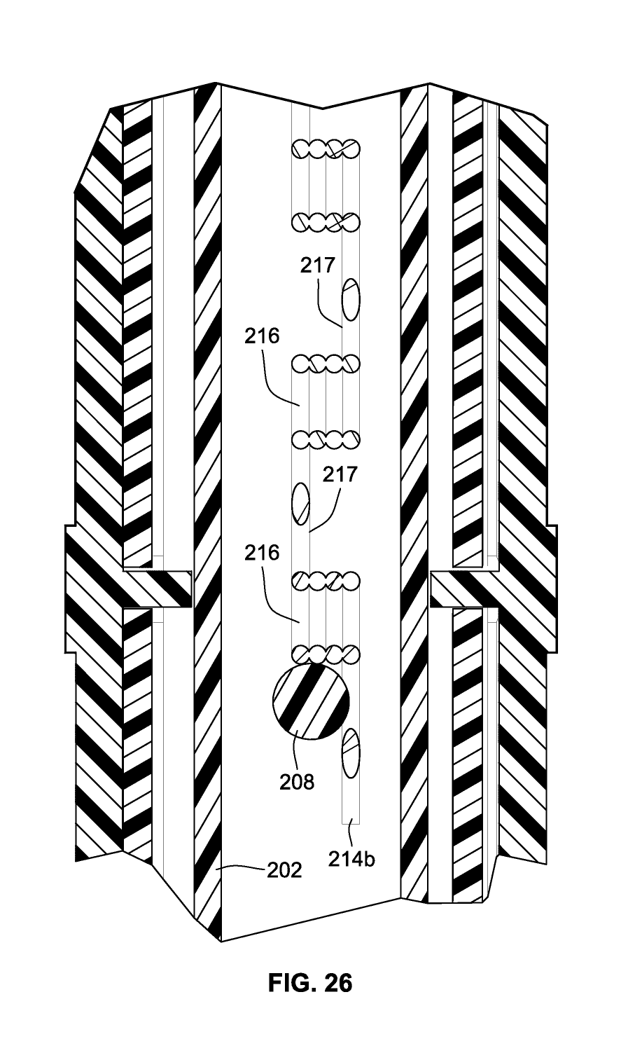

Another embodiment, illustrated in FIGS. 20-26, replaces the actuators 40 and 100 with a housing section 200 made of a flexible and resilient material so that it can be deformed inwardly against the crests 203a and 203b of a serpentine element 201, which is also made of a flexible and resilient material. The serpentine element 201 has opposite end tabs 201 a and 201b with respective apertures 202a and 202b for attachment to a blade carrier 202 and to a housing 210, respectively. Specifically, the aperture 202a fits over a post 37 on the housing 210, and the aperture 202b fits over a post 208 on a blade carrier 202. Thus, when the serpentine element 201 is extended by pressing down on the crests 203a and 203b of the serpentine element 201, the blade carrier 202 is moved relative to the housing section 210, thereby advancing the blade 20 out of the housing 210.

To advance the blade carrier 202, the user simply squeezes the flexible housing section 200 inwardly against the crests 203a and 203b of the serpentine element 201. This reduces the height of the crests, which in turn elongates the serpentine element 201 to push the carrier 202 forwardly relative to the housing section 210, thereby advancing the blade 20 to its exposed position outside the housing. The blade 20 will remain in this advanced position as long as sufficient pressure is maintained on the resilient housing section 200 to maintain the serpentine element 201 in its elongated condition. When that pressure is released, the resilient serpentine element 201 returns to its original shape, which tends to pull the carrier 202 back to its retracted position. The biasing spring 36 ensures that the carrier 202 is quickly retracted, by augmenting the retracting force applied to the carrier by the serpentine element 201.

In order to maintain the central valley portion 203c of the serpentine element 201 (between the two crests 203a and 203b) in the same plane as the two end tabs 201a and 201b, a pair of bosses 204 project laterally from opposite sides of the center of the valley portion 203c. These bosses 204 fit into a pair of channels 212 formed in the ribs of the carrier 202. This permits the bosses to move longitudinally within the channels 212 while maintaining the valley portion 203c at a fixed elevation, so that the elongation of the serpentine element 201 is dependent entirely on the degree of deformation of the two crests 203a and 203b.

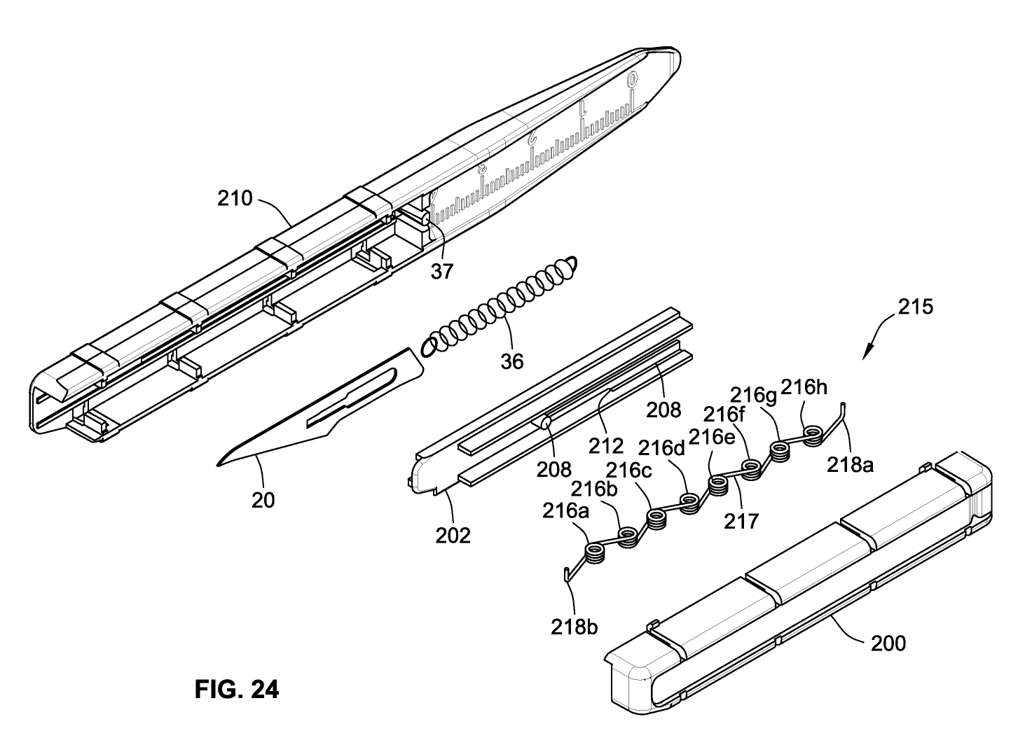

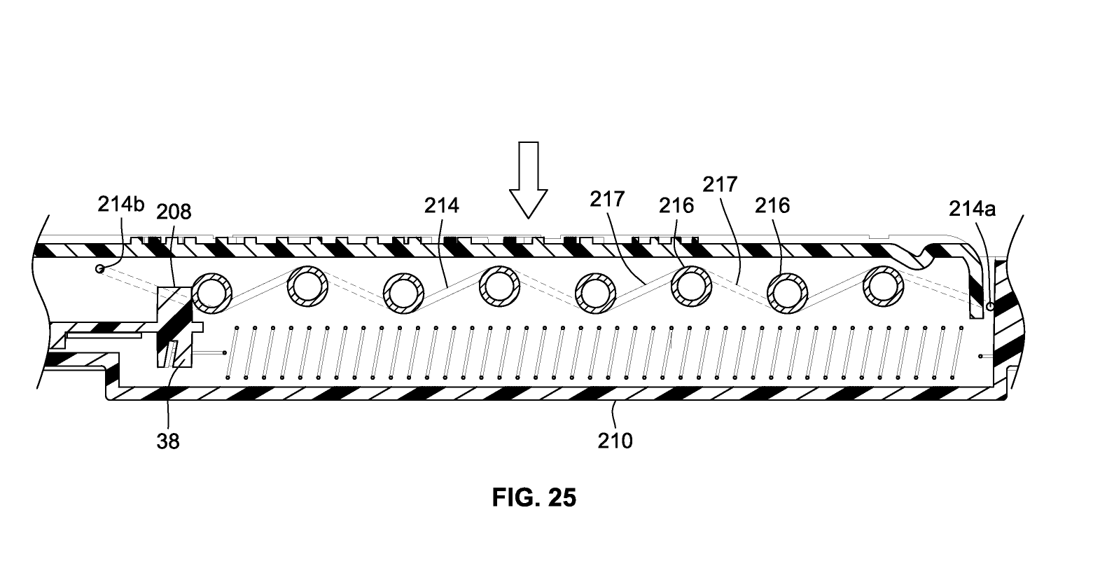

In a further modified embodiment illustrated in FIGS. 24-26, the serpentine element is in the form of a multi-coil spring 215 that is both flexible and resilient. The spring 215 is formed by a single wire that forms multiple coils 216a-216h interconnected by linear wire segments 217 to form a general serpentine shape. Bent end portions 218a and 218b are formed on opposite ends of the spring 215 to attach the spring 215 to the housing 210 at one end and to the carrier 202 at the other end. When the flexible housing section 200 is deformed inwardly against the crests formed by alternate ones of the coils 216a-216h (see FIG. 25), the overall length of the multi-coil spring 215 increases, which causes the spring 215 to push the blade carrier 202 forwardly, thereby advancing the blade 20 to its exposed position outside the housing. The blade 20 will remain in this advanced position as long as sufficient pressure is maintained on the resilient housing section 200. When that pressure is released, the spring 215 returns to its original shape, which tends to pull the carrier 202 back to its retracted position. The biasing spring 36 ensures that the carrier 202 is quickly retracted, by augmenting the retracting force applied to the carrier by the spring 215.

While particular embodiments and applications of the present invention have been illustrated and described, it is to be understood that the invention is not limited to the precise construction and compositions disclosed herein and that various modifications, changes, and variations may be apparent from the foregoing descriptions without departing from the spirit and scope of the invention as defined in the appended claims.

1. A concealed-blade scalpel comprising
an elongated housing forming an interior cavity and an opening at one end of said housing to provide access to said interior cavity,
a surgical cutting blade mounted within said cavity for sliding movement relative to said housing so that said blade can be moved between (1) a retracted position in which the cutting edge of said blade is located entirely within said cavity, and (2) an advanced position in which the cutting edge of said blade is positioned outside said housing to permit said blade to be used for cutting,
a biasing element mounted within said cavity and coupled to said blade and said housing for urging said blade toward said retracted position, and
an actuator coupled to said blade for moving said blade to said advanced position in response to manual pressure applied to said actuator, said biasing element urging said blade toward said retracted position so that said blade is automatically moved to said retracted position when said manual pressure applied to said actuator is removed.

2. The concealed-blade scalpel of claim 1 in which said actuator is a slide coupled to said blade and accessible on the exterior of said housing to permit manual engagement by a user for moving said blade from said retracted position to said advanced position, against the urging of said blade toward said retracted position by said biasing element.

3. The concealed-blade scalpel of claim 2 in which said slide has a gripping surface to facilitate manual manipulation of said slide.

4. The concealed-blade scalpel of claim 2 in which said slide is mounted on a first side of said housing, and a second side of said housing, opposite said first side, also forms a gripping surface.

5. The concealed-blade scalpel of claim 1 in which said actuator includes a flexible, resilient element that is deformable in response to gripping pressure applied to said housing to move said blade to said advanced position, against the urging of said blade toward said retracted position by said biasing element.

6. The concealed-blade scalpel of claim 5 in which said actuator is a portion of said housing that is flexible and resilient so that it can be deformed into engagement with said flexible, resilient element by a user to permit said blade to be manually moved from said retracted position to said advanced position by gripping pressure applied to said deformable portion of said housing.

7. The concealed-blade scalpel of claim 5 in which said flexible, resilient element has a serpentine shape.

8. The method of claim 1 in which said actuator is coupled to said housing for sliding movement in response to a manual force applied to said actuator, said actuator being coupled to said blade for advancing said blade in response to pivoting movement of said actuator.

9. The method of claim 1 in which said actuator is mounted for pivoting movement on said housing in response to a manual force applied to said actuator, said actuator being coupled to said blade for advancing said blade in response to pivoting movement of said actuator.

10. The method of claim 1 which includes a blade carrier coupling said actuator to said blade and mounted for movement within said housing to move said blade between said retracted and advanced positions.

11. The method of claim 10 in which said blade carrier is slidably mounted within said housing.

12. The method of claim 1 in which said biasing element continuously urges said blade toward said retracted position.

13. The method of claim 1 in which said blade is mounted on a movable carrier, and said biasing element acts on said carrier to urge said blade toward said retracted position.

14. The method of claim 1 in which said biasing element is a coil spring that expands when said blade is moved from said retracted position to said advanced position.

15. A method of concealing the blade of a scalpel, comprising
mounting a scalpel blade for sliding movement within an interior cavity in an elongated housing that forms an opening at one end of said housing to provide access to said interior cavity,
applying a manual force to slide said blade between (1) a retracted position in which the cutting edge of said blade is located entirely within said cavity, and (2) an advanced position in which the cutting edge of said blade is advanced through said opening and is positioned outside said housing to permit said blade to be used for cutting, while continuously biasing said blade toward said retracted position so that said blade is automatically moved to said retracted position when said manual force is removed.

16. The method of claim 15 in which said blade is mounted on a carrier contained within said cavity and coupled to a manually operated actuator on the exterior of said housing, said biasing force is applied to said carrier by a spring within said housing, and said manual force is applied to said actuator to move said carrier to slide said blade between said retracted and advanced positions.
